(19) United States
(12) Patent Application Publication
(10) Pub. No.: US 2005/0238638 A1
(43) Pub. Date: Oct. 27, 2005

(54) Dietary Supplement that Serves as a Carbohydrate Blocker and Hangover Remedy

(76) Inventor: Jonathan Gutwein, Englewood, NJ (US)

Correspondence Address: JEFFREY FURR, 253 N. MAIN STREET, JOHNSTOWN, OH, 43031, US

(21) Appl. No.: 10/709,214

(22) Filed: Apr. 21, 2004

(51) Int. Cl. A61K 33/44; A61K 9/48; A61K 35/78

(52) U.S. Cl. 424/125; 424/757; 424/725.1

(57) ABSTRACT

The present invention relates to a dietary supplement that serves as a carbohydrate blocker and as a hangover remedy. The serving size is 2 capsules. These are formed by combining and pressing into capsules the following materials with the preferred quantity: Chromium picolinate 120 mcg, White Kidney Bean Extract (Phaseolus Vulgaris) 1000 mg, Vanadium chelate 400 mcg, Calcium Carbonate 150 mg, Vegetable Carbon 300 mg, Vitamin B-12 900 mcg, Vitamin B2 6 mg, and Coenzyme Q10 100 mg.

### BACKGROUND OF INVENTION

This invention relates to a dietary supplement more particularly a dietary supplement that serves as a carbohydrate blocker and hangover blocker.

1. Background

Given today's push for low carbohydrate diets there exists a need to block carbohydrates from being absorbed by the body. The current popularity of the Atkin's Diet is pushing people to look for a low carbohydrate diet and lifestyle.

There are also needs for a supplement that will assist someone with the pains of a hangover. Hangovers have been a problem for mankind for untold millennium. It has cost many hours of lost work time and productivity. In short, there is a need for a product that blocks carbohydrates and prevent hangovers for beer drinkers and other high carb alcoholic beverages. Currently, there is no existing product on the market that combines both benefits/ingredients in one product. There is no product that can currently do both.

2. Prior Art

U.S. Pat. No. 6,034,125 by McLeod and issued on Mar. 7, 2000, is for a method of treating depression using chromium. It discloses a method directed to a treatment of depression in men and women by administering to a patient a therapeutically effective amount of chromium in a pharmaceutically acceptable form. Chromium, preferably in the form of chromium picolinate, is administered to the patient at dosages in a preferred range of about 200 to about 500 micrograms chromium.

U.S. Pat. No. 5,612,039 by Policappelli, et al. and issued on Mar. 18, 1997, is for a dietary supplement. It discloses a composition for dietary supplementation includes the following three compositions: The first composition is Germander (Teucrium Chamaedrys Herba), Camelia Tea (Camelia Thea Folia), and Spirulina (Spirulina Maxima); the second composition is Garcinia Gambogia, dry extract (Garcinia Cambogia Fructuse), Brown Algae, dry extract (Fucus Vesicolosus), and Germander (Teucrium Chamaedrys Herba); the third composition is Garcinia Gambogia, dry extract (Garcinia Cambogia Fructuse), Brown Algae, dry extract (Fucus Vesicolosus), and Orthosiphon, dry extract (Orthosiphon Stamineus Folia), respectively. Additionally, there is a fourth composition including Common Bean (Phaseolus Vulgaris Fructus), Garcinia Cambogia, dry extract (Garcinia Cambogia Fructus), Pineapple, dry extract (Ananas Sativus Stipites), Gymnema Sylvestre, dry extract(Gymnema Sylvestre Folia), and Chromium Dinicotinate (Chromium).

U.S. patent application 20030211172 by Jones, et al. and published on Nov. 13, 2003, is for a composition and method for substantially reducing the deleterious effects of alcohol on the body. It discloses a composition and method that are provided for substantially preventing hangovers. The composition includes an effective amount of amino acids glycine and Glutathione, B vitamins and at least one mineral compound including each of magnesium, selenium, and molybdenum. Other vitamins, mineral compounds, flavorings, coloring, and solubility agents may also be added.

### SUMMARY OF INVENTION

The present invention relates to a dietary supplement that serves as a carbohydrate blocker and as a hangover remedy. The serving size is 2 capsules. These capsules are formed by combining and pressing into capsules the following materials with the preferred quantity: Chromium picolinate 120 mcg, White Kidney Bean Extract (Phaseolus Vulgaris) 1000 mg, Vanadium chelate 400 mcg, Calcium Carbonate 150 mg, Vegetable Carbon 300 mg, Vitamin B-12 900 mcg, Vitamin B2 6 mg, and Coenzyme Q10 100 mg.

### BRIEF DESCRIPTION OF DRAWINGS

Without restricting the full scope of this invention, the preferred form of this invention is illustrated in the following drawings:

FIG. 1 shows the basic components of the invention.

### DETAILED DESCRIPTION

The following description is demonstrative in nature and is not intended to limit the scope of the invention or its application or uses.

There are a number of significant design features and improvements incorporated within the invention.

The present invention relates to a dietary supplement that serves as a carbohydrate blocker and as a hangover remedy. The three main ingredients of the supplement are white kidney bean, vegetable carbon, and calcium carbonate.

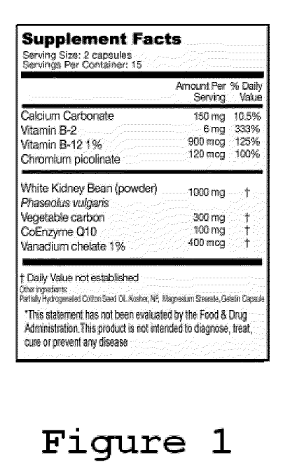

In the preferred embodiment, the supplement is formed by combining and pressing into capsules the following materials with the preferred quantity: Chromium picolinate 120 mcg, White Kidney Bean Extract (Phaseolus Vulgaris) 1000 mg, Vanadium chelate 400 mcg, Calcium Carbonate 150 mg, Vegetable Carbon 300 mg, Vitamin B-12 900 mcg, Vitamin B2 6 mg, and Coenzyme Q10 100 mg. This is shown in FIG. 1.

The components are mixed in power form. The composition is mixed by blending all components/ingredients without flavorings (including but not limited to sugar).

The final product yield is calculated by weight and staged for packaging. It is compressed into red and white gel caps. Equivalent materials can be substituted as well as different amounts of the components can be used for much of the same effect. The product and formula can also be produced in the form of gelatin capsules, pills, resins or any other compressed material. The product can be produced as a powder, liquid, syrup and any other available substance to produce finished food product.

In the preferred embodiment the serving size is 2 capsules. The supplement will be shipped in bottles that contain 30 capsules with cotton and a moisture tag and then double sealed (inside and out). They are in a cobalt blue PETE bottle.

This supplement will not prevent intoxication and is not intended to treator prevent excessive alcohol consumption. The supplement should be taken prior to the ingestion of the food and/or drink that the user wants to block. If this is a beer or ale it should be taken orally prior to drinking the drink.

The white kidney bean extract is used as a carbohydrate blocker while the useof carbon, vegetable carbon and calcium carbonate is used to block the hangover.

The compounds used are as follows: White Kidney Bean Extract (Phaseolus Vulgaris) is a known carbohydrate blocker. It disrupts the breakdown of carbs and stops simple sugars from being converted into fat.

Carbon and Vegetable carbon are used to absorb toxins.

Calcium carbonate is the active ingredient in most calcium supplements and antacids.

Chromium Picolinate aids in glucose tolerance by production of glucose tolerance factor (GTF). By aiding in this function, it minimizes the ability of alcohols to adversely effect glucose/insulin tolerance and may possibly reduce alcohol aggravated diabetes.

Cobalamin (Vitamin B-12) works in conjunction with Vitamins B-1 and B-6 to reduce alcohol induced neuralgias.

Pyridoxine (Vitamin B-6), alcohol has been shown to cause B-6 depletion. Therefore it is important to adjunct the composition with vitamin B-6. B-6 depletion causes nausea, depression, vomiting, and peripheral neuritis.

Coenzyme Q₁₀ (also known as CoQ₁₀, Q₁₀, vitamin Q₁₀, ubiquinone, or ubidecarenone) is a compound that is made naturally in the body. It is a substance needed for the proper functioning of an enzyme, a protein that speeds up the rate at which chemical reactions take place in the body. The Q and the 10 in coenzyme Q refer to parts of the compound's chemical structure.

Vanadium, as in Vanadium chelate, is an ultra-trace mineral found in the human diet and the human body. It is essential for some animals. Deficiency symptoms in these animals include growth retardation, bone deformities, and infertility. However, vanadium has not been proven to be an essential mineral for humans. Vanadium may play a role in building bones and teeth.

The current invention is a product that combines white kidney bean for carbohydrate blocking which is combined with one of a number of variety of other (whole sentence too awkward) ingredients known to prevent hangovers including, but not only, the carbons and calcium carbonate mentioned above.

### CONCLUSION

The invention is a great supplement to block the absorbance of carbohydrates which is helpful with low carbohydrate diets. This also has an additional advantage of reducing the effects of a hangover.

Although the present invention has been described in considerable detail with reference to certain preferred versions thereof, other versions are possible. Therefore, the point and scope of the appended claims should not be limited to the description of the preferred versions contained herein.

As to a further discussion of the manner of usage and operation of the present invention, the same should be apparent from the above description. Accordingly, no further discussion relating to the manner of usage and operation will be provided.

With respect to the above description, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Furthermore, since numerous modifications and changes will readily occur to those skilled in the art, it is not desired to limit the invention to the exact construction and operation shown and described, and accordingly, all suitable modifications and equivalents may be resorted to, falling within the scope of the invention.

1. A supplement comprising:
a carbohydrate blocker and a hangover preventer.

2. A supplement according to claim 1 in which said carbohydrate block is Phaseolus Vulgaris.

3. A supplement according to claim 1 in which said hangover preventer selected from a group consist of one or more of a set of carbon, vegetable carbon and calcium carbonate.

4. A supplement according to claim 1 in which said carbohydrate block is Phaseolus Vulgaris and said hangover preventer consists of one or more of a set of carbon, vegetable carbon and calcium carbonate.

5. A supplement according to claim 1 in which are formed in capsules.

6. A supplement comprising:
white kidney bean extract, carbon, and calcium carbonate.

7. A supplement according to claim 6 further comprising having said carbon being vegetable carbon.

8. A supplement according to claim 6 further comprising having Vanadium chelate.

9. A supplement according to claim 6 further comprising having Calcium Carbonate.

10. A supplement according to claim 6 further comprising having Vitamin B-12.

11. A supplement according to claim 6 further comprising having Vitamin B2.

12. A supplement according to claim 6 further comprising having Coenzyme Q10.

13. A supplement according to claim 6 in which are formed in cpasules

14. A supplement according to claim 13 in said capsules are ingested prior to use.

14. A supplement comprising:
Chromium picolinate, White Kidney Bean Extract (Phaseolus Vulgaris), Vanadium chelate, Calcium Carbonate, Vegetable Carbon, Vitamin B-12, Vitamin B2, and Coenzyme Q10.

15. A supplement according to claim 15 comprising: 120 mcg of Chromium picolinate, 1000 mg of White Kidney Bean Extract (Phaseolus Vulgaris), 400 mcg Vanadium chelate, 150 mg Calcium Carbonate, 300 mg Vegetable Carbon, 900 mcg Vitamin B-12, 6 mg Vitamin B2, 100 mg Coenzyme Q10.

17. A supplement according to claim 15 in which are formed in capsules.

18. A supplement according to claim 17 in said capsules are ingested prior to use.
